(19) United States
(12) Patent Application Publication
(10) Pub. No.: US 2010/0114244 A1
(43) Pub. Date: May 6, 2010

(54) ELECTRICAL RENAL AUTONOMIC BLOCKADE

(75) Inventors: Venkatesh Manda, Stillwater, MN (US); Kenneth M. Anderson, Bloomington, MN (US); David E. Euler, Plymouth, MN (US); Avram Scheiner, Vadnais Heights, MN (US)

Correspondence Address: Medtronic, Inc., 710 Medtronic Parkway, Minneapolis, MN, 55432, US

(73) Assignee: Medtronic, Inc.

(21) Appl. No.: 12/263,202

(22) Filed: Oct. 31, 2008

(51) Int. Cl. A61N 1/36; A61N 1/08

(52) U.S. Cl. 607/60; 607/62

(57) ABSTRACT

Electrical stimulation may be configured to decrease renal sympathetic activity by creating at least a partial functional conduction block in the efferent and/or afferent sympathetic nerve fibers that innervate the kidneys. An electrical stimulator may deliver a stimulation signal to a renal nerve of a patient. The stimulation signal may be a biphasic signal with a frequency of approximately 100 hertz to 20 kilohertz. In some examples, a sensor may sense a physiological parameter of the patient, and the stimulation generator may activate, deactivate, or adjust the stimulation signal based on the physiological parameter. The physiological parameter may be indicative of sympathetic activity within the patient.

### TECHNICAL FIELD

The disclosure relates to medical devices and, more particularly, medical devices that deliver electrical stimulation.

### BACKGROUND

A wide variety of implantable medical devices for delivering a therapy or monitoring a physiologic condition of a patient have been clinically implanted or proposed for clinical implantation in patients. Some implantable medical devices may employ one or more elongated electrical leads and/or sensors. Such implantable medical devices may deliver therapy or monitor the heart, muscle, nerve, brain, stomach or other organs. In some cases, implantable medical devices may deliver electrical stimulation therapy and/or monitor physiological signals via one or more electrodes or sensor elements, which may be included as part of one or more elongated implantable medical leads. Implantable medical leads may be configured to allow electrodes or sensors to be positioned at desired locations for delivery of stimulation or sensing electrical signals. For example, electrodes or sensors may be located at a distal portion of the lead. A proximal portion of the lead may be coupled to an implantable medical device housing, which may contain electronic circuitry such as stimulation generation and/or sensing circuitry.

In patients with heart failure or hypertension, renal sympathetic activity has been shown to be markedly elevated. Elevated renal sympathetic activity may result in renal vasoconstriction, increased retention of sodium, as well as an increase in release of renin and angiotensin. Increased sodium, rennin and angiotensin in turn further exacerbate heart failure and hypertension by increasing blood volume and arterial hypertension, and triggering signs and symptoms of cardio-pulmonary congestion, such as edema or peripheral fluid accumulation. Furthermore, chronic elevation of renal sympathetic tone in various disease states, i.e., with our without heart failure, may play a role in the development of overt renal failure and end-stage renal disease.

Efforts to control renal sympathetic activity have included administration of medications such as angiotensin-converting enzyme inhibitors angiotensin II receptor blockers and beta-blockers. Such medications may have a broader effect than controlling the renin angiotensin aldosterone system (RAAS). Furthermore, symptoms associated with elevated renal sympathetic activity may persist despite such medications.

### SUMMARY

In general, the disclosure relates to delivering electrical stimulation to decrease renal sympathetic activity. Renal sympathetic activity may worsen symptoms of heart failure, hypertension, and/or chronic renal failure. For example, renal sympathetic activity may increase fluid retention by the kidneys, which in turn increases blood volume, arterial hypertension, and pulmonary congestion. Electrical stimulation may be configured to decrease renal sympathetic activity by creating at least a partial functional conduction block in the efferent and/or afferent sympathetic nerve fibers that innervate the kidneys.

In some examples, a sensor may sense a physiological parameter of the patient, and the stimulation generator may activate, deactivate, or adjust the stimulation signal based on the physiological parameter. The physiological parameter may be indicative of sympathetic activity within the patient. Examples of physiological parameters that may indicate the level of sympathetic activity within the patient include blood pressure, blood flow, vascular tone, plasma renin level, or norepinephrine level. The parameters may be measured proximate to the renal system, such as a renal artery blood pressure or blood flow, or elsewhere within the patient. In some examples, information regarding the physiological parameter, or information derived therefrom, such as information regarding the progression or status of heart failure, renal failure, hypertension, or autonomic tone, may be transmitted to an external device, such as a programmer or server, for presentation to a clinician or other user.

In one aspect, the disclosure is directed to a method comprising sensing a physiological parameter of a patient, generating a stimulation signal via an implantable electrical stimulator based on the physiological parameter, delivering the stimulation signal from the implantable stimulator to a renal nerve of the patient, transmitting information regarding the physiological parameter to an external device outside of the patient, and presenting the information to a user.

In another aspect, the disclosure is directed to a system comprising an implantable sensor that senses a physiological parameter of a patient, an implantable electrical stimulator that communicates with the implantable sensor, wherein the implantable electrical stimulator comprises a stimulation generator that generates a stimulation signal based on the physiological parameter and delivers the stimulation signal to a renal nerve of the patient, and an external device that receives information regarding the physiological parameter and presents the information to a user.

In another aspect, the disclosure is directed to a method comprising sensing a physiological parameter indicative of sympathetic activity within a patient, identifying an increase in sympathetic activity based on the physiological parameter, and delivering a stimulation signal to a renal nerve of the patient in response to the increase in sympathetic activity.

In another aspect, the disclosure is directed to a system comprising a sensor that senses a physiological parameter indicative of sympathetic activity within a patient, a processor that identifies an increase in sympathetic activity based on the physiological parameter, and an electrical stimulator that delivers a stimulation signal to a renal nerve of the patient in response to the increase in sympathetic activity.

In another aspect, the disclosure is directed to a method for inhibiting renal sympathetic activity comprising generating a biphasic stimulation signal with a frequency of approximately 100 hertz to approximately 20 kilohertz and delivering the stimulation signal to a renal nerve of the patient.

In another aspect, the disclosure is directed to a system comprising a signal generator that generates a biphasic stimulation signal with a frequency of approximately 100 hertz to approximately 20 kilohertz and an electrode configured to be positioned proximate to a renal nerve of the patient, wherein the signal generator delivers the stimulation signal to the renal nerve via the electrode.

The details of one or more examples of the disclosure are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example therapy system that delivers stimulation therapy to decrease renal sympathetic activity.

FIG. 2 is a conceptual diagram illustrating the therapy system of FIG. 1 in greater detail.

FIG. 3 is a functional block diagram of an example implantable medical device.

FIG. 4 is a block diagram of an example medical device programmer.

FIG. 5 is a flow diagram illustrating an example technique for delivering electrical stimulation to a patient to decrease renal sympathetic activity.

FIG. 6 is a flow diagram illustrating an example technique for modifying stimulation delivery based on a sensed physiological parameter.

FIG. 7 is a flow diagram illustrating an example technique for sensing physiological parameters.

FIG. 8 is a block diagram illustrating an example system that includes an external device, such as a server, and one or more computing devices that are coupled to the medical device and programmer shown in FIG. 1 via a network.

### DETAILED DESCRIPTION

In patients with heart failure, hypertension, and chronic renal failure, renal sympathetic activity may be elevated. As one example, the decreased cardiac output that results from heart failure may decrease circulation to the kidneys. The kidneys are responsible for maintaining blood volume and may perceive the decreased circulation as a decrease in blood volume. To counteract the perceived decrease in blood volume, the renal system may increase renal sympathetic activity. Renal sympathetic activity increases sodium retention to increase blood volume and increases the release of renin and angiotensin to increase blood pressure. The increased blood volume and blood pressure may further exacerbate heart failure and hypertension as well as trigger signs and symptoms of cardio-pulmonary congestion. As the symptoms of heart failure worsen, the renal system may respond by further increasing renal sympathetic activity.

Electrical stimulation may be configured to decrease renal sympathetic activity by creating at least a partial functional conduction block in the efferent and/or afferent sympathetic nerve fibers that innervate the kidneys. The blockade may reversibly interrupt neural signals between the central nervous system and the renal nerves that innervate the kidneys. For example, when an implantable medical device (IMD) delivers electrical stimulation, renal sympathetic tone may be reduced. Renal sympathetic tone may return when the IMD ceases stimulation delivery. In some examples, the conduction of sympathetic neural signals between the central nervous system and the renal nerves is substantially completely blocked when the IMD delivers electrical stimulation. Reducing renal sympathetic activity may increase renal blood flow, increase renal sodium excretion, and/or decrease renin release from the kidneys.

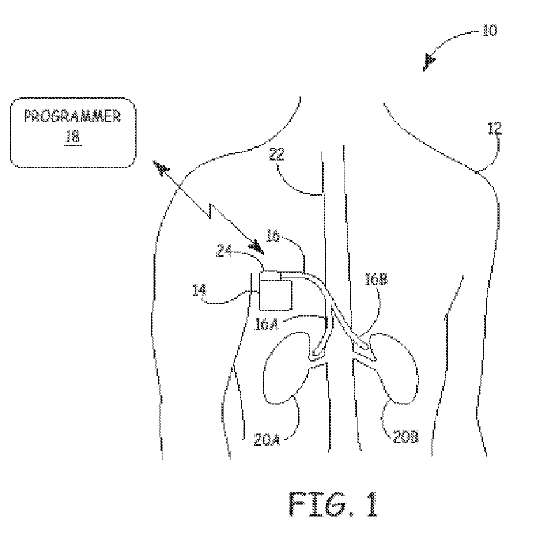

FIG. 1 is a conceptual diagram illustrating an example therapy system 10 that provides stimulation therapy to patient 12. Patient 12 ordinarily, but not necessarily, will be a human. Therapy system 10 includes an IMD 14, which is coupled to lead 16 and programmer 18. In the example illustrated in FIG. 1, lead 16 is bifurcated into two distal segments, i.e., branches, 16A and 16B. In other examples, lead 16 may be unbranched. Although IMD 14 is illustrated in the example of FIG. 1, in other examples an external medical device positioned outside of patient 12 may provide the functionality of IMD 14.

IMD 14 may generate and deliver electrical stimulation e.g., in the form of electrical pulses or a substantially continuous signal, to decrease renal sympathetic activity of patient 12. For example, IMD 14 may generate and deliver stimulation to a nerve or other tissue site of patient 12, e.g., proximate to kidneys 20A and 20B (collectively “kidneys 20”), via one or more electrodes (not shown in FIG. 1) carried on distal segments 16A and 16B of lead 16 and/or one or more electrodes on an outer housing of IMD 14. The renal nerves that innervate kidneys 20 may exit spinal cord 22 proximate to kidneys 20. IMD 14 may generate and deliver stimulation to a renal nerve proximate to spinal cord 22 and/or kidneys 20 to decrease renal sympathetic activity.

In the example shown in FIG. 1, electrodes on distal portions 16A and 16B of lead 16 are positioned to deliver bilateral electrical stimulation, e.g., to nerves that innervate both kidneys 20. IMD 14 may deliver the same or different therapy to kidneys 20A and 20B. For example, IMD 14 may sense parameters indicative of renal sympathetic activity proximate to each of kidneys 20A and 20B and separately control stimulation delivery via each of distal segments 16A and 16B of lead 16 based on the sensed parameters. In other example therapy systems, IMD 14 may be coupled to two or more leads, e.g., furcated and/or non-furcated leads, either directly or indirectly, e.g., via a lead extension. For example, IMD 14 may be coupled to two non-furcated leads such that a distal end of one lead is positioned proximate to kidney 20A and the distal end of the other lead is positioned proximate to kidney 20B. As another example, IMD 14 may be coupled to a single unbranched lead for applications in which IMD 14 delivers unilateral stimulation to a single side of patient 12, e.g., to the nerves that innervate either kidney 20A or kidney 20B.

IMD 14 may sense electrical signals associated with the sympathetic activity of the nerves that innervate kidneys 20 via electrodes carried by lead 16. For example, IMD 14 may monitor an electrogram (EGM) signal of a renal nerve to determine the level of renal sympathetic activity within patient 12. In some examples, IMD 14 may monitor separate signals from distal segments 16A and 16B of lead 16 to evaluate renal sympathetic activity proximate to kidneys 20A and 20B individually. The configurations of electrodes used by IMD 14 for sensing may be unipolar or bipolar.

Additionally or alternatively, therapy system 10 may include other sensors (not shown in FIG. 1) to monitor sympathetic activity, e.g., systemic and/or renal sympathetic activity. For example, therapy system 10 may include one or more intravascular sensors in communication with IMD 14. Intravascular sensors, such as chemical sensors, may monitor the blood chemistry of patient 12, e.g., a plasma renin level or norepinephrine level within the blood of patient 12. Other intravascular sensors, such as a strain gauge, capacitive pressure sensor, ultrasonic flow sensor, or electrodes for determining impedance, may monitor blood pressure, blood flow, and/or assess vascular tone in a blood vessel of patient 12.

Although intravascular sensors are described primarily herein, therapy system 10 may include any appropriate intravascular or extravascular sensor to detect these or any other physiological parameters indicative of sympathetic activity. For example, one or more sensors may be implanted in extravascular spaces of patient 12, such as the intraperitoneal space within the abdominal cavity of patient 12. Sensors implanted in extravascular spaces may permit monitoring of blood parameters without requiring intravascular implantation.

The sensors of therapy system 10 may monitor systemic sympathetic activity and/or renal sympathetic activity. For example, a sensor may be implanted within the superior vena cava that supplies blood to the heart of patient 12 to monitor systemic sympathetic activity of patient 12. In contrast, a sensor may be implanted within a renal vessel, e.g., a renal artery or renal vein, to monitor renal sympathetic activity. Sensors positioned proximate to a renal nerve of patient 12, e.g., proximate to kidneys 20 in the abdomen of patient 12, may monitor renal sympathetic tone. Sensors that monitor renal sympathetic tone may provide IMD 14 with more specific feedback than sensors that monitor systemic sympathetic tone.

In the example of FIG. 1, IMD 14 has been implanted in the chest cavity of patient 12. Other implant locations are also contemplated, such as in the back or abdominal cavity of patient 12. IMD 14 may be, for example, subcutaneously or submuscularly implanted in the body of patient 12 at any appropriate location. Upon implantation of IMD 14, the proximal end of lead 16 may be both electrically and mechanically coupled to connector 24 of IMD 14 either directly or indirectly, e.g., via a lead extension. In particular, conductors disposed in the lead body of lead 16 may electrically connect stimulation electrodes (and sense electrodes, if present) of lead 16 to IMD 14.

In some examples, external programmer 18 may be a handheld computing device or a computer workstation. Programmer 18 may include a user interface that receives inputs from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 18 may additionally or alternatively include a peripheral pointing device, such as a mouse, via which a user may interact with the user interface. In some examples, a display of programmer 18 may include a touch screen display, and a user may interact with programmer 18 via the display.

A user, such as patient 12, a physician, technician, or other clinician, may interact with programmer 18 to communicate with IMD 14. The user may interact with programmer 18 to retrieve physiological or diagnostic information from IMD 14. For example, the user may use programmer 18 to retrieve information from IMD 14 regarding sensed physiological parameters of patient 12 indicative of renal sympathetic activity, such as electrical signals, e.g., EGM signals, blood pressure, or the like. IMD 14 may transfer information to programmer 18 regarding diagnostic information determined based on the sensed physiological parameters, such as renal function and heart failure status, for view by a user, e.g., a clinician and/or patient 12. A user may also interact with programmer 18 to program IMD 14, e.g., select values for operational parameters of IMD 14 based on the sensed physiological parameters received from IMD 14.

The user may use programmer 18 to program therapy parameters for electrical stimulation. The therapy parameters may include an electrode combination for delivering stimulation signals, as well as an amplitude, which may be a current or voltage amplitude, and, if IMD 14 delivers electrical pulses, a pulse width, and a pulse rate for stimulation signals to be delivered to patient 12. The electrode combination may include a selected subset of one or more electrodes located on implantable lead 16 coupled to IMD 14 and/or a housing of IMD 14. The electrode combination may also refer to the polarities of the electrodes in the selected subset. By selecting particular electrode combinations, a clinician may target particular anatomic structures within patient 12, such as the renal nerves. In addition, by selecting values for amplitude, pulse width, and pulse rate, the physician can attempt to generate an efficacious therapy for patient 12 that is delivered via the selected electrode subset.

As another example, the user may use programmer 18 to retrieve information from IMD 14 regarding the performance or integrity of IMD 14 or other components of therapy system 10, such as lead 16 or a power source of IMD 14. With the aid of programmer 18 or another computing device, a user may select values for therapy parameters for controlling therapy delivery by IMD 14. The values for the therapy parameters may be organized into a group of parameter values referred to as a “therapy program” or “therapy parameter set.” “Therapy program” and “therapy parameter set” are used interchangeably herein.

Programmer 18 may communicate with IMD 14 via wireless communication using any techniques known in the art. Examples of communication techniques may include, for example, low frequency or radiofrequency (RF) telemetry, but other techniques are also contemplated. In some examples, programmer 18 may include a programming head that may be placed proximate to the patient's body near the IMD 14 implant site in order to improve the quality or security of communication between IMD 14 and programmer 18.

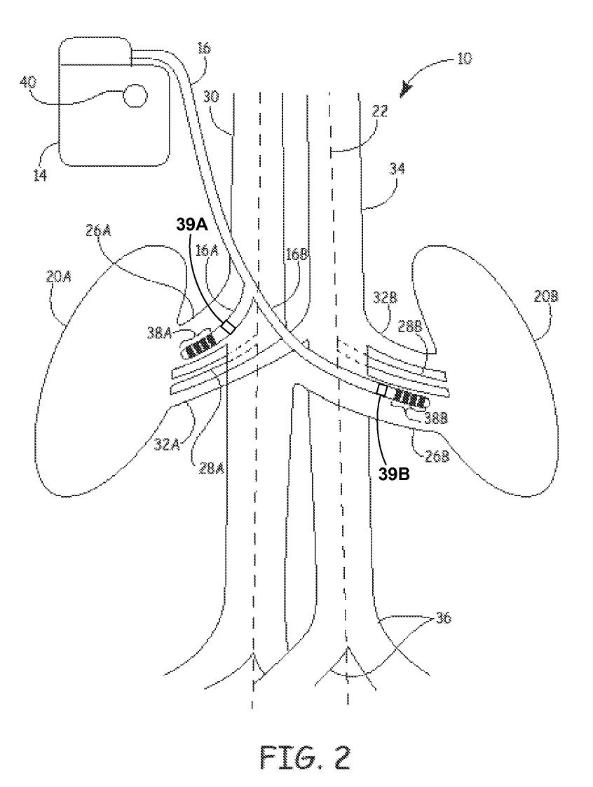

FIG. 2 is a conceptual diagram illustrating IMD 14 and lead 16 of therapy system 10 in greater detail. In the example illustrated in FIG. 2, distal segment 16A of lead 16 is implanted in left renal vein 26A, and distal segment 16B of lead 16 is implanted in right renal vein 26B. In this manner, electrodes 38A on distal segment 16A may deliver stimulation to renal nerves 28A, and electrodes 38B on distal segment 16B may deliver stimulation to renal nerves 28B.

Renal nerves 28A and 28B (collectively “renal nerves 28”) illustrate the approximate location of the nerves that innervate kidneys 20. For example, renal nerves 28 may exit spinal cord 22 approximately at the level of kidneys 20 and may approach kidneys 20 in a similar manner as renal arteries 32A and 32B (collectively “renal arteries 32”) and renal veins 26 (collectively “renal veins 26”). Renal nerves 28 may lie directly adjacent to renal arteries 32. Renal nerves 28, as described herein, may refer to the renal plexus as a whole, any individual nerve of the renal plexus, and/or any other nerve that innervates kidneys 20.

To aid in positioning distal segments 16A and 16B proximate to renal nerves 28A and 28B, respectively, lead 16 may be inserted into inferior vena cava 30. Once lead 16 is inserted into inferior vena cava 30, distal segment 16A may be guided into left renal vein 26A using a first guidewire and distal segment 16B may be guided into right renal vein 26B using a second guidewire. Methods and systems for guiding distal segments of a bifurcated lead to different tissue sites are described in further detail in U.S. Pat. No. 7,142,919 to Hine at el., which issued on Nov. 28, 2006 and is entitled, “RECONFIGURABLE FAULT TOLERANT MULTIPLE-ELECTRODE CARDIAC LEAD SYSTEM,” and is incorporated herein by reference in its entirety.

Although distal segments 16A and 16B are implanted within renal veins 26 in the example of FIG. 2, in other examples distal segments 16A and 16B may be implanted at any other location proximate to renal nerves 28. As one example, distal segments 16A and 16B may be implanted within renal arteries 32A and 32B, respectively. In some examples, lead 16 may be inserted through one of common iliac arteries 36 that branch off of abdominal aorta 34 to allow guidewires to direct distal segments 16A and 16B to renal arteries 32A and 32B. Since renal nerves 28 may lie adjacent to renal veins 26 and renal arteries 32, implanting distal segments 16A and 16B within renal veins 26 and/or renal arteries 32 may allow IMD 14 to stimulate renal nerves 28.

Renal veins 26 are larger than renal arteries 32 and may allow for easier intravascular lead implantation compared to renal arteries 32. On the other hand, renal arteries 32 may be located closer than renal veins 26 to renal nerves 28. Thus, stimulation from electrodes implanted within renal veins 26 may more easily capture renal nerves 28.

In the example illustrated in FIG. 2, distal segment 16A includes one or more electrodes 38A and distal segment 16B includes one or more electrodes 38B. Electrodes 38A may be ring electrodes that extend substantially completely around the circumference of distal segment 16A or partial ring electrodes that extend partially around the circumference of the distal segment 16A. Partial ring electrodes may be useful in directing electrical stimulation in a particular direction, e.g., toward renal nerves 28A. Electrodes 38B may also be ring or partial ring electrodes. The number, configuration, and type of electrodes 38A and 38B (collectively “electrodes 38”) illustrated in FIG. 2 are merely exemplary. Other examples may include any configuration, number, or type of electrodes 38.

IMD 14 may also include one or more housing electrodes, such as housing electrode 40, which may be formed integrally with an outer surface of a hermetically-sealed housing of IMD 14 or otherwise coupled to the housing of IMD 14. In some examples, housing electrode 40 is defined by an uninsulated portion of an outward facing portion of the housing of IMD 14. In some examples, housing electrode 40 comprises substantially all of the IMD housing. Other divisions between insulated and uninsulated portions of the housing may be employed to define two or more housing electrodes.

IMD 14 may deliver electrical stimulation to renal nerves 28 via any combination of electrodes 38 and housing electrode 40, e.g., any unipolar or multipolar electrode configuration, to decrease renal sympathetic activity. Each of electrodes 38 may be individually activated by IMD 14 to deliver stimulation using a variety of electrode configurations. In some examples, IMD 14 may deliver a stimulation signal between one of electrodes 38 and housing electrode 40, i.e., in a unipolar configuration. As another example, IMD 14 may deliver a stimulation signal between a plurality of electrodes 38, e.g., in a multipolar configuration. IMD 14 may deliver the same or different stimulation signal to both sets of electrodes 38A and 38B, i.e., to deliver bilateral stimulation. As another example, IMD 14 may deliver a stimulation signal to one set of electrode 38A and 38B, i.e., to deliver unilateral stimulation.

Distal segments 16A and/or 16B may include one or more fixation elements to prevent migration of distal segments 16A and/or 16B. For example, distal segment 16A may include an expandable fixation element, e.g., an expandable stent or cage. The expandable fixation element may be inserted into inferior vena cava 30 in an unexpanded configuration and expanded to engage an inner surface of renal vein 26A once distal segment 16A is properly placed within renal vein 26A. The fixation element may fixate distal segment 16A within renal vein 26A without impeding blood flow within renal vein 26A.

In some examples, the fixation element may be conductive and IMD 14 may use the fixation element as an electrode to stimulate renal nerve 28A. In other examples, the fixation element may include a plurality of electrically isolated conductive portions such that IMD 14 may independently activate the various conductive portions as electrodes for sensing and/or stimulation. Distal segment 16B may also include a fixation element to fixate distal segment 16B within renal vein 26B. Although expandable fixation elements are described for purposes of example, distal segments 16A and 16B may include any appropriate type of fixation element. Additionally, the fixation elements may be sized and configured to fixate distal segments 16A and 16B in other vessels of patient 12, e.g., renal arteries 32.

In other examples, distal segments 16A and/or 16B may be positioned extravascularly. For example, electrodes 3 8A and 3 8B of distal segments 16A and 16B may be included on cuff electrode assemblies that wrap at least partially around renal nerves 28A and 28B, respectively. Since renal nerves 28 may include fibers that run in close proximity to renal veins 26 and renal arteries 32, the cuff electrode assemblies may be implanted around renal veins 26 and/or renal arteries 32 instead of directly around renal nerves 28. A cuff electrode assembly may include a U-shaped cross section configured to fit about a selected portion of the circumference of a nerve, e.g., one of renal nerves 28, or vessel, e.g., one of renal veins 26 or renal arteries 32. A cuff electrode assembly may also include one or more conductive portions that serve as electrodes 38. Examples of cuff electrode assemblies are described in U.S. Pat. No. 5,344,438 to Testerman et al., which issued on Sep. 4, 1994 and is entitled, “Cuff Electrode,” and is incorporated herein by reference in its entirety.

In yet other examples, distal segments 16A and/or 16B may be transvascularly positioned renal nerves 28A and 28B. For example, electrodes 38A and 38B of distal segments 16A and 16B may be positioned extravascularly although other portions of distal segments 16A and 16B may be implanted intravascularly. As one example, lead 16 may be inserted into superior vena cava 30, distal segment 16A may be guided into right renal vein 26A, and distal segment 16B may be guided into left renal vein 26B. The portions of distal segments 16A and 16B carrying electrodes 38A and 38B may be guided through walls of respective veins 26 such that electrodes 38A and 38B are positioned extravascularly proximate to renal nerves 28A and 28B, respectively. Transvascular implantation of electrodes is described in further detail in U.S. patent application Ser. No. 10/411,891 by Lamson et al., which was filed on Apr. 11, 2003, is entitled, “Devices and Methods for Transluminal or Transthoracic Interstitial Electrode Placement,” and is incorporated herein by reference in its entirety.

IMD 14 may sense electrical signals attendant to the sympathetic activity of renal nerves 28 that innervate kidneys 20 via electrodes 38 and/or housing electrode 40. For example, IMD 14 may monitor electrogram (EGM) signals of renal nerves 28 to determine the level of renal sympathetic activity within patient 12. In some examples, IMD 14 may monitor separate signals from one or more of electrodes 38A on the right side of patient 12 and one or more of electrodes 38B on the left side of patient 12 to evaluate renal sympathetic activity associated with kidneys 20A and 20B individually. The configurations of electrodes used by IMD 14 for sensing may be unipolar or bipolar.

Additionally or alternatively, lead 16 may include other sensors to monitor physiological parameters indicative of sympathetic tone. In the example illustrated in FIG. 2, distal segment 16A of lead 16 includes sensor 39A proximate to kidney 20A, and distal segment 16B of lead 16 includes sensor 39B proximate to kidney 20B. Other examples any include any number or configuration of sensors 39. For example, lead 16 may include one or more chemical sensors 39 to monitor blood chemistry, e.g., to monitor norepinephrine levels and/or plasma renin levels, within patient 12. Lead 16 may also include one or more sensors 39 to detect blood pressure, blood flow, and/or assess vascular tone within one or more vessels, e.g., inferior vena cava 30, renal veins 26, renal arteries 32, and/or common iliac arteries 36, of patient 12. In some examples, sensors 39 are positioned within renal vessels, e.g., renal veins 26 and/or renal arteries 32, or otherwise proximate to kidneys 20 to monitor renal sympathetic activity. Monitoring renal sympathetic tone may provide more specific feedback to IMD 14 than monitoring systemic sympathetic tone. In some examples, therapy system 10 may include sensors 39 that are intravascularly implanted within patient 12 but not carried by lead 16. Such sensors may be in wired and/or wireless communication with IMD 14.

In some examples, therapy system 10 includes one or more sensors 39 implanted in extravascular spaces, such as the intraperitoneal space within the abdominal cavity, of patient 12. Sensors 39 implanted in extravascular spaces may permit monitoring of blood parameters without requiring intravascular implantation. Such sensors 39 may be in wired and/or wireless communication with IMD 14. In examples in which lead 16 is implanted extravascularly, these sensors 39 may be carried by lead 16.

IMD 14 may use the physiological signals sensed by electrodes 38 and/or other intravascular and/or extravascular sensors 39 to control stimulation delivery to renal nerves 28. For example, IMD 14 may initiate, modify, or cease stimulation delivery based on one or more sensed physiological parameters. As one example, IMD 14 may identify an increase in sympathetic activity based on one or more sensed physiological parameters and deliver a stimulation signal to renal nerves 28 in response to the increase in sympathetic activity. For example, IMD 14 may initiate stimulation delivery or modify the stimulation parameters in response to the detected increase in sympathetic activity. IMD 14 may modify one or more stimulation parameters, e.g., electrode configuration, amplitude, pulse width, and/or pulse rate, to increase the intensity of the stimulation signal in response to the detected increase in sympathetic activity.

IMD 14 may identify a level of sympathetic activity based on one or more physiological signals from one or more sensors, e.g., electrodes 38 or sensors 39. For example, IMD 14 may identify an increase in sympathetic activity by detecting an increase in plasma renin levels, e.g., within a renal vessel, and deliver a stimulation signal in response to the detection. IMD 14 may monitor the magnitude and time-course of changes in one or more physiological signals, such as plasma renin levels, renal blood flow, or other biomarkers, to identify changes in sympathetic activity of patient 12. IMD 14 may monitor the magnitude and time-course of changes in one or more physiological signals while IMD 14 delivers stimulation to decrease renal sympathetic activity to investigate the effectiveness of stimulation delivery and/or the effectiveness of the physiological signals in measuring the effectiveness of stimulation delivery.

In some examples, IMD 14 may use two or more physiological signals to monitor the sympathetic activity of patient 12. As one example, IMD 14 may monitor a norepinephrine level with the patient's blood, e.g., within a renal vessel. Elevated norepinephrine levels may indicate elevated sympathetic activity. If the norepinephrine level rises above a threshold, IMD 14 may monitor renal blood flow and/or renal blood pressure, e.g., within renal arteries 32. Since sympathetic efferent activation causes renal vasoconstriction and a reduction in renal blood flow, blood flow and/or blood pressure in a renal vessel may indicate the level of renal sympathetic activity. If blood flow to kidneys 20 is decreased and/or renal blood pressure is increased, IMD 14 may identify an increase in sympathetic activity and deliver a stimulation signal to renal nerves 28. Once the blood flow and/or blood pressure return to normal, IMD 14 may switch back to monitoring norepinephrine levels. The methods of sensing physiological parameters and identifying increases in sympathetic activity described herein are merely examples.

In other examples, IMD 14 may utilize a plurality of sensors, e.g., electrodes 38 and/or sensors 39, in complimentary and/or orthogonal manners to detect changes in sympathetic activity and regulate stimulation delivery. For example, IMD 14 may sense a first physiological parameter. When the first physiological parameter indicates increased sympathetic activity, IMD 14 may sense a second physiological parameter. The second physiological parameter may be used to confirm the increase in sympathetic activity. For example, IMD 14 may only identify an increase in sympathetic activity when both the first and second physiological parameters indicate increased sympathetic activity.

In general, IMD 14 may identify changes in the sympathetic activity level of patient 12 based on one or more sensed physiological parameters and control stimulation delivery to renal nerves 28 in response to the identified changes. In some examples, the sensed physiological parameters indicate renal sympathetic activity, and IMD 14 identifies changes in renal sympathetic activity. In some examples, IMD 14 may maintain sympathetic activity below a threshold level by adjusting stimulation delivery based on the sensed sympathetic physiological parameters. IMD 14 may use the sensed physiological parameters to determine when patient 12 requires stimulation and the minimum level of stimulation required to maintain renal sympathetic activity below a desired level. IMD 14 may sense physiological parameters on the right and left sides of patient 12, e.g., proximate to kidneys 20A and 20B, and/or control stimulation delivery to the right and left sides of patient 12, e.g., renal nerves 28A and 28B, individually.

Additionally or alternatively, IMD 14 may control stimulation delivery based on parameters other than sensed physiological parameters. For example, IMD 14 may deliver stimulation during specific portions of the day, e.g., according to a schedule. The intensity of the stimulation may be preprogrammed, e.g., via programmer 18, or responsive to sensed physiological parameters. As one example, a schedule may specify therapy intensities and/or therapy parameters for certain portions of the day instead of or in addition to specifying which portions of the day IMD 14 delivers stimulation. Alternatively, patient 12 may activate IMD 14, e.g., via programmer 18, to deliver stimulation when needed. Again, the intensity of the stimulation may be preprogrammed, e.g., via programmer 18, or responsive to sensed physiological parameters.

In some examples, IMD 14 may modify one or more stimulation parameters over time to prevent or minimize accommodation. For example, IMD 14 may deliver stimulation signals using different electrode combinations, waveforms, amplitudes, or frequencies to prevent or minimize accommodation. In examples in which IMD 14 delivers electrical pulses, IMD 14 may also deliver signals with different pulse widths and/or pulse rates to prevent or minimize accommodation.

In some examples, IMD 14 delivers a high frequency, biphasic stimulation signal to renal nerves 28 to decrease renal sympathetic activity. For example, IMD 14 may generate a stimulation signal with a frequency of approximately 100 hertz to approximately 20 kilohertz and deliver the stimulation signal to renal nerves 28 of patient 12. In some examples, the stimulation signal may have a frequency of approximately 100 hertz to approximately 10 kilohertz. In other examples, IMD 14 may generate a stimulation signal with a frequency of approximately 2 kilohertz or higher. The stimulation signal may have a voltage amplitude of approximately 0.5 volts to approximately 20 volts and, in some examples, a voltage amplitude of approximately 0.5 volts to approximately 10 volts. Alternatively, the stimulation signal may have a current amplitude of approximately 1 to approximately 12 milliamperes. A biphasic stimulation signal has portions with opposite polarities, e.g., positive and negative portions.

High-frequency biphasic electrical stimulation may create a reversible functional conduction block in the efferent and afferent nerve fibers that innervate kidneys 20, e.g., renal nerves 28. High-frequency biphasic electrical stimulation may be effective in producing reversible nerve conduction block in unmyelinated nerve fibers, such as the unmyelinated post-ganglionic nerve fibers of the renal sympathetic nerves. Biphasic electrical stimulation may also be charge-balanced, and thereby prevent and/or reduce corrosion of electrodes 38.

IMD 14 may use alternating current (AC) to deliver stimulation signals to reduce renal sympathetic activity. High-frequency AC stimulation has been shown to produce block of nerve conduction in motor nerves and may also be effective at producing conduction block in renal sympathetic nerves, e.g., renal nerves 28. IMD 14 may also use monopolar and/or multipolar electrode configurations to achieve at least partial conduction block in renal nerves 28. Example stimulation waveforms that IMD 14 may utilize to achieve at least partial renal nerve blockage include sinusoidal waveforms, square waveforms, and other continuous time signals. As an alternative, IMD 14 may deliver stimulation in the form of pulses.

In some examples, IMD 14 delivers high voltage stimulation in addition to or as an alternative to high frequency stimulation. High voltage stimulation may use voltages significantly higher than the physiological voltages renal nerves 28 use to conduct neural signals. For example, IMD 14 may deliver high voltage stimulation at approximately 15 volts or higher. High voltage stimulation may stun renal nerves 28 and at least partially prevent renal nerves 28 from conducting neural signals. High voltage stimulation may utilize direct current (DC) signals and may be configured to minimize damage to renal nerves 28.

As another example, IMD 14 may deliver stimulation to create a unidirectional or collision block. In this manner, IMD 14 may deliver stimulation signals that propagate in a direction that opposes the efferent neural signals traveling toward kidneys 20. The stimulation signals delivered by IMD 14 may collide with the neural signals traveling from the central nervous system of patient 12 to kidneys 20 and at least partially prevent conduction of the efferent neural signals. IMD 14 may configure at least some of electrodes 38 as anodes and cathodes to achieve collision block in renal nerves 28. In other examples, IMD 14 may deliver stimulation signal that propagate in a direction that opposes the afferent neural signals traveling from kidneys 20 to the central nervous system to at least partially block the afferent neural signals from reaching the central nervous system of patient 12.

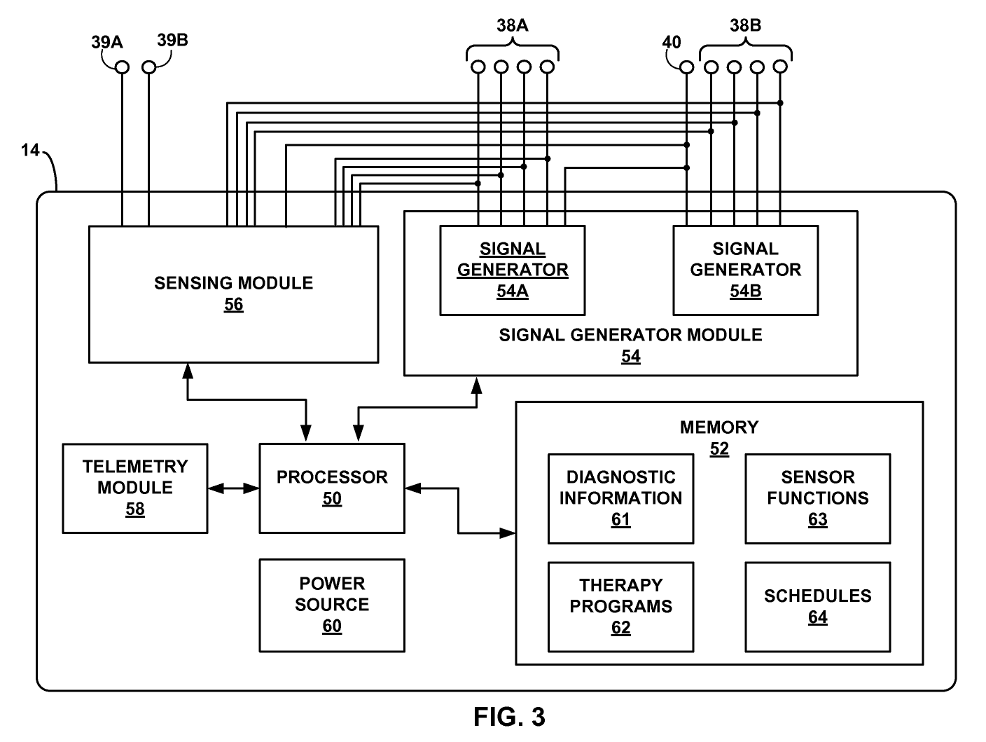

FIG. 3 is a functional block diagram illustrating various components of IMD 14 according to one example. In the example of FIG. 3, IMD 14 includes processor 50, memory 52, signal generator module 54, sensing module 56, telemetry module 58, and power source 60. Telemetry module 58 may permit communication with programmer 18 to receive, for example, new therapy programs or adjustments to therapy programs. Telemetry module 58 may also permit communication with programmer 18 to transfer, for example, sensed physiological parameters to programmer 18.

Memory 52 includes computer-readable instructions that, when executed by processor 50, cause IMD 14 and processor 50 to perform various functions attributed to IMD 14 and processor 50 herein. Memory 52 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. As described in further detail below, memory 52 may store, for example, diagnostic information 61 regarding sensed physiological parameters, therapy programs 62 defining therapy parameters for stimulation delivery, sensor functions 63 including instructions for sensing sympathetic activity of patient 12, and/or schedules 64 that define when to deliver stimulation therapy.

Processor 50 may include any one or more of a microprocessor, a controller, a digital signal processor (DSP), an application specific integrated circuit (ASIC), a field-programmable gate array (FPGA), or equivalent discrete or integrated logic circuitry. In some examples, processor 50 may include multiple components, such as any combination of one or more microprocessors, one or more controllers, one or more DSPs, one or more ASICs, or one or more FPGAs, as well as other discrete or integrated logic circuitry. The functions attributed to processor 50 herein may be embodied as software, firmware, hardware or any combination thereof.

Processor 50 controls operation of IMD 14, e.g., controls signal generator module 54 to deliver stimulation therapy according to a selected one or more therapy programs 62, which may be stored in memory 52. For example, processor 50 may control signal generator module 54 to deliver electrical signals with current or voltage amplitudes, pulse widths (if applicable), and rates specified by one or more stimulation programs 62. Processor 50 may also control signal generator module 54 to deliver the stimulation signals via subsets of the electrodes 38 and 40 with polarities, the subsets and polarities specified as electrode combinations or configurations by one or more therapy programs 62. In some examples, signal generator module 54 includes two stimulation generators 54A and 54B to deliver separate stimulation signals to renal nerves 28A and renal nerves 28B, respectively. Processor 50 may control signal generators 54A and 54B to deliver the same or different stimulation signals to renal nerves 28A proximate to kidney 28A on the right side of patient 12 and renal nerves 28B proximate to kidney 28B on the left side of patient 12. Processor 50 may also include separate circuitry to separately control stimulation delivery to renal nerves 28A and 28B.

In some examples, processor 50 may control signal generator module 54 to deliver stimulation signals to patient 12 based on physiological parameter values sensed by sensing module 56, which may indicate a level of sympathetic activity that patient 12 is experiencing. In other examples, processor 50 may control signal generator module 54 to deliver stimulation signals to patient 12 according to one or more predetermined schedules 64 that are independent of physiological parameter values sensed by sensing module 56. The schedules 64 may be determined by a clinician and stored in memory 52. The schedules 64 may indicate times that IMD 14 should initiate, increase, decrease, and/or cease stimulation delivery.

Sensing module 56 may monitor signals from at least two of electrodes 38 and 40 to monitor electrical activity of renal nerves 28, via electrogram (EGM) signals. Sensing module 96 may also include a switch module to select the available electrodes 38 and 40 that are used to sense the electrical activity of renal nerves 28. In some examples, processor 50 may select the electrodes 38 and 40 that function as sense electrodes via the switch module within sensing module 56, e.g., by providing signals via a data/address bus. For example, processor 50 may access sensing functions 63 stored in memory 52 and select a plurality of electrodes 38 and 40 to function as sense electrodes based on sensing functions 63. In some examples, sensing module 56 includes one or more sensing channels, each of which may comprise an amplifier. In response to the signals from processor 50, the switch module within sensing module 56 may couple the outputs from the selected electrodes 38 and 40 to one of the sensing channels.

Sensing module 56 may also receive signals from other sensors 39 in wired communication with IMD 14, and telemetry module 58 may receive signals from sensors in wireless communication with IMD 14. For example, sensing module 56 may receive signals from non-electrode sensors 39, e.g., chemical, pressure, and/or flow sensors, coupled to lead 16. Telemetry module 58 may receive signals from any sensors in wireless communication with IMD 14 and may provide the received data to processor 50 and/or sensing module 56. Processor 50 may control sensing module 56 and/or telemetry module 58 to retrieve sensed physiological signals based on sensing functions 63 stored in memory 52. Sensing functions 63 may define which sensors are activated to identify changes in the sympathetic activity level of patient 12 and/or the values of sensed physiological parameters that indicate elevated sympathetic activity.

Telemetry module 58 may also permit IMD 14 to transmit information regarding the physiological parameters, e.g., sensed physiological parameters, information regarding renal function, and/or heart failure status, to an external device such as programmer 18 for view by a clinician, patient 12, and/or another user. In some examples, memory 52 stores diagnostic information 61, e.g., information regarding the physiological parameters, and telemetry module 58 may retrieve diagnostic information 61 from memory 52 for transmission to an external device such as programmer 18.

Telemetry module 58 includes any suitable hardware, firmware, software or any combination thereof for communicating with another device, such as programmer 18 (FIG. 1) or sensors. Under the control of processor 50, telemetry module 58 may receive downlink telemetry from and send uplink telemetry to programmer 18 with the aid of an antenna, which may be internal and/or external. Processor 50 may provide the data to be uplinked to programmer 18 and the control signals for the telemetry circuit within telemetry module 58, e.g., via an address/data bus. In some examples, telemetry module 58 may provide received data to processor 50 via a multiplexer.

The various components of IMD 14 are coupled to power source 60, which may include a rechargeable or non-rechargeable battery or a supercapacitor. A non-rechargeable battery may be selected to last for several years, while a rechargeable battery may be inductively charged from an external device, e.g., on a daily or weekly basis. In some examples, power source 60 recharge via induction or ultrasonic energy transmission, and include an appropriate circuit for recovering transcutaneously received energy. For example, power source 60 may be coupled to a secondary coil and a rectifier circuit for inductive energy transfer.

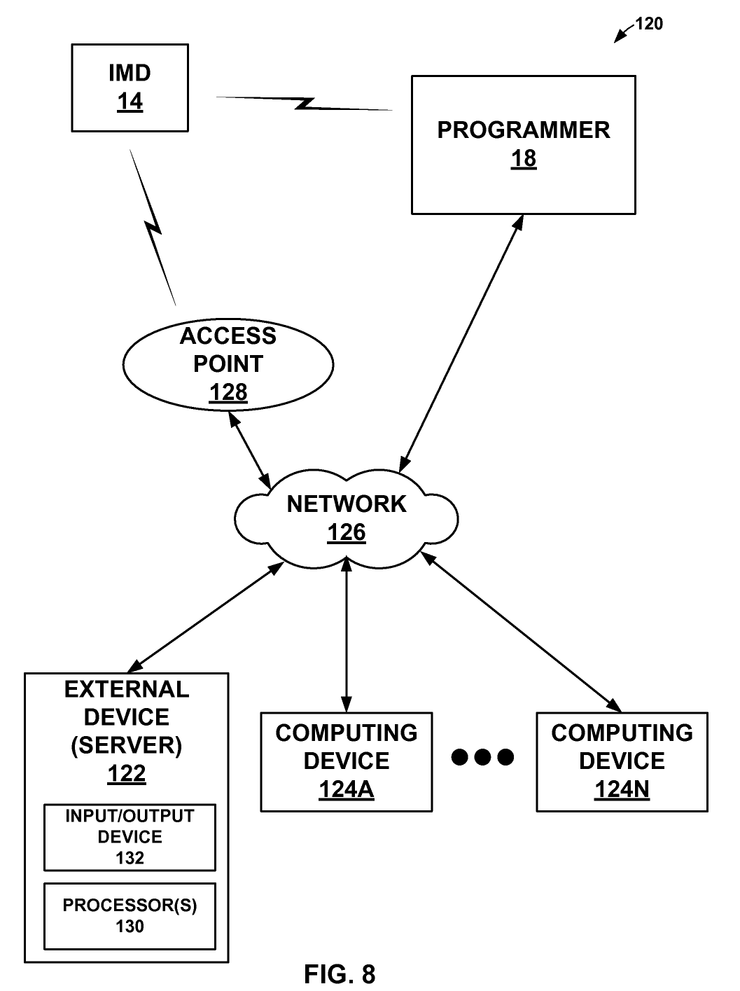

As described in further detail with respect to FIG. 8, in some examples data generated by sensing module 56 and stored in memory 52 may be uploaded to a remote server, from which a clinician, patient or another user may access the data to, for example, evaluate the progression or heart failure, renal failure, or hypertension. An example of a remote server includes the CareLink Network, available from Medtronic, Inc. of Minneapolis, Minn. An example system may include an external device, such as a server, and one or more computing devices that are coupled to IMD 14 and programmer 18 via a network.

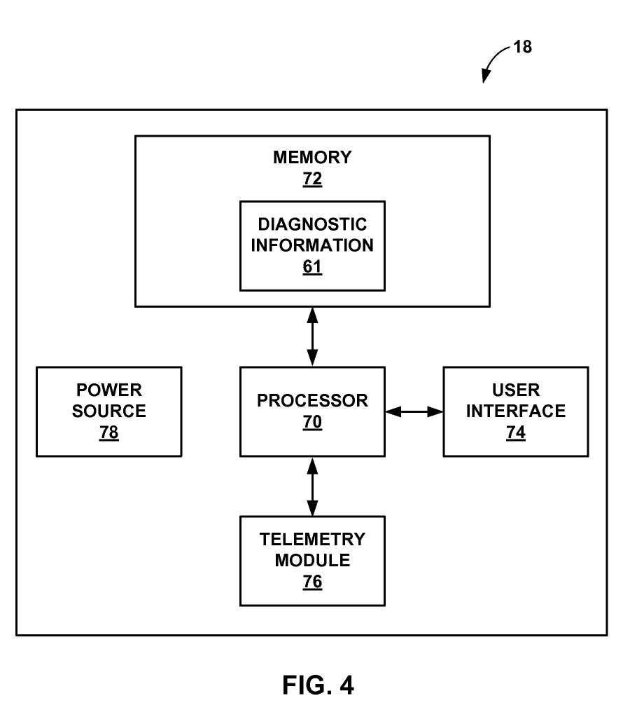

FIG. 4 is a block diagram of an example medical device programmer 18. As shown in FIG. 4, programmer 18 includes processor 70, memory 72, user interface 74, telemetry module 76, and power source 78. Programmer 18 may be a dedicated hardware device with dedicated software for programming of IMD 14. Alternatively, programmer 18 may be an off-the-shelf computing device running an application that enables programmer 18 to program IMD 14.

A user, e.g., clinician, may use programmer 18 to select therapy programs (e.g., sets of stimulation parameters), generate new therapy programs, modify therapy programs through individual or global adjustments or transmit the new programs to IMD 14 (FIG. 1). The user may program, modify or control any aspect of the operation of IMD 14 via programmer 18. For example, the user may modify therapy programs 62, sensor functions 63, or schedules 64. In this manner, the user may modify the manner in which stimulation is delivered to the renal nerves, including the timing and intensity of such stimulation, the manner in which physiological parameters indicative of sympathetic activity are sensed, and the manner in which the stimulation is delivered based on the sensed parameters. The user may interact with programmer 18 via user interface 74, which may include a display to present a graphical user interface to a user, and a keypad or another mechanism for receiving input from a user.

Furthermore, the user may view information via user interface 74. For example, a user may view diagnostic information 61 collected by IMD 18, or other sensors. As indicated in FIG. 4, memory 72 of programmer 18 may store diagnostic information 72. Based on such information, a user may evaluate the progression or status of a patient condition, such as heart failure, renal failure, or hypertension.

Processor 70 can take the form one or more microprocessors, DSPs, ASICs, FPGAs, programmable logic circuitry, or the like, and the functions attributed to processor 70 herein may be embodied as hardware, firmware, software or any combination thereof. Memory 72 may store instructions that cause processor 70 to provide the functionality ascribed to programmer 18 herein, and information used by processor 70 to provide the functionality ascribed to programmer 18 herein. Memory 72 may include any fixed or removable magnetic, optical, or electrical media, such as RAM, ROM, CD-ROM, hard or floppy magnetic disks, EEPROM, or the like. Memory 72 may also include a removable memory portion that may be used to provide memory updates or increases in memory capacities. A removable memory may also allow patient data to be easily transferred to another computing device, or to be removed before programmer 18 is used to program therapy for another patient. Memory 72 may also store information that controls therapy delivery by IMD 14, such as stimulation parameter values. For example, memory 72 may store therapy programs 62, which telemetry module 76 may access and transmit to IMD 14.

Programmer 18 may communicate wirelessly with IMD 14, such as using RF communication or proximal inductive interaction. This wireless communication is possible through the use of telemetry module 76, which may be coupled to an internal antenna or an external antenna. An external antenna that is coupled to programmer 18 may correspond to the programming head that may be placed proximate to the patient's body near the IMD 14 implant site. Telemetry module 76 may be similar to telemetry module 58 of IMD 14 (FIG. 3).

Telemetry module 76 may also be configured to communicate with another computing device via wireless communication techniques, or direct communication through a wired connection. Examples of local wireless communication techniques that may be employed to facilitate communication between programmer 18 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 18 without needing to establish a secure wireless connection.

Power source 78 delivers operating power to the components of programmer 18. Power source 78 may include a battery and a power generation circuit to produce the operating power. In some examples, the battery may be rechargeable to allow extended operation. Recharging may be accomplished by electrically coupling power source 78 to a cradle or plug that is connected to an alternating current (AC) outlet. In addition or alternatively, recharging may be accomplished through proximal inductive interaction between an external charger and an inductive charging coil within programmer 18. In other examples, traditional batteries (e.g., nickel cadmium or lithium ion batteries) may be used. In addition, programmer 18 may be directly coupled to an alternating current outlet to power programmer 18. Power source 78 may include circuitry to monitor power remaining within a battery. In this manner, user interface 74 may provide a current battery level indicator or low battery level indicator when the battery needs to be replaced or recharged. In some cases, power source 78 may be capable of estimating the remaining time of operation using the current battery.

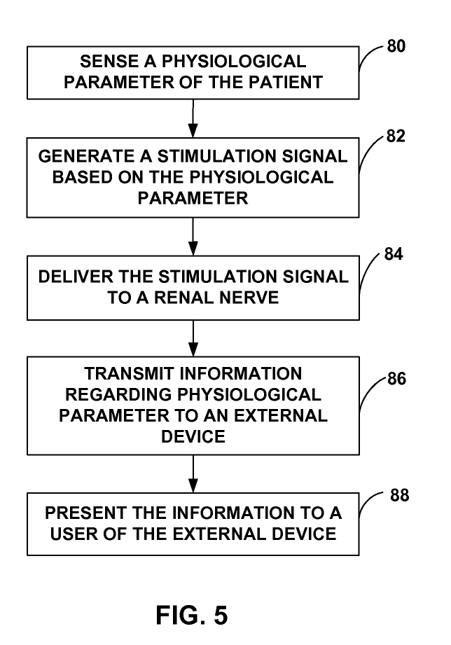

FIG. 5 is a flow diagram illustrating an example technique for delivering electrical stimulation to a patient to decrease renal sympathetic activity. Sensing module 56 of IMD 14 may sense a physiological parameter of patient 12 (80). For example, processor 50 may control IMD 14 to sense a physiological parameter of patient 12 via any combination of electrodes 38 and 40. As another example, sensing module 56 may sense one or more physiological parameters via non-electrode sensors 39, e.g., chemical or mechanical sensors, coupled to IMD 14 via lead 16. Additionally or alternatively, telemetry module 58 of IMD 14 may receive one or more physiological signals from sensors in wireless communication with IMD 14.

Signal generator 54 of IMD 14 may generate a stimulation signal based on the sensed physiological parameter (82). For example, the physiological parameter may be indicative of sympathetic activity, e.g., systemic or renal sympathetic activity, within patient 12. Processor 50 within IMD 14 may identify an increase in sympathetic activity based on the physiological parameter and generate the stimulation signal in response to the increase in sympathetic activity.

Alternatively, telemetry module 58 of IMD 14 may transmit information regarding the sensed physiological parameter to an external device outside of patient 12, e.g., programmer 18. Processor 70 of programmer 18 may analyze the information regarding the physiological parameter to identify changes in sympathetic activity within patient 12. In response to an increase in sympathetic activity, programmer 18 may direct signal generator 54 of IMD 14 to generate a stimulation signal (82). Processor 70 of programmer 18 may identify increases in sympathetic activity in examples in which IMD 14 does not include circuitry to perform the analysis of the physiological parameter, e.g., due to size constraints of IMD 14. In other examples in which processor 50 of IMD 14 identifies increases in sympathetic activity, telemetry module 58 may transmit information regarding the physiological parameter to programmer 18 or another external device for viewing by a user, e.g., to supplement the automatic identification of increased sympathetic activity performed by processor 50 of IMD 14.

IMD 14 may deliver the stimulation signal to one or more of renal nerves 28 (84). For example, IMD 14 may deliver the stimulation signal to one or more of renal nerves 28 in response to the increase in sympathetic activity within patient 12. The stimulation signal may be configured to decrease renal sympathetic activity within patient 12. In one example, IMD 14 delivers the stimulation signal to renal nerves 28A via electrodes 38A carried by distal segment 16A of lead 16. Additionally or alternatively, IMD 14 may deliver the stimulation signal to renal nerves 28B via electrodes 38B carried by distal segment 16B of lead 16. IMD 14 may deliver unilateral stimulation, e.g., to either renal nerves 28A on the right side of patient 12 or renal nerves 28B on the left side of patient 12, or bilateral stimulation, e.g., to both renal nerves 28A on the right side of patient 12 and renal nerves 28B on the left side of patient 12. In examples in which IMD 14 delivers bilateral stimulation, IMD 14 may deliver the same or different stimulation signals to renal nerves 28A and 28B.

In some examples, the stimulation signal may be a high frequency, biphasic stimulation signal. High-frequency biphasic electrical stimulation may create a reversible functional conduction in renal nerves 28. Biphasic electrical stimulation may also prevent and/or reduce corrosion of electrodes 38. As one example, the stimulation signal may be a biphasic stimulation signal with a frequency of approximately 100 hertz to approximately 20 kilohertz.

As previously described, telemetry module 58 of IMD 14 may transmit information regarding the physiological parameter to programmer 18 or another external device (86), and the external device may present the information to a user, e.g., patient 12 or a clinician, for viewing (88). For example, telemetry module 58 may transmit information regarding the physiological parameter itself, e.g., blood pressure and plasma renin level. As another example, telemetry module 58 may transmit other diagnostic information based on the sensed physiological parameter, such as renal function and heart failure status. The user may interpret the information and provide programming instructions to IMD 14, e.g., to improve therapy effectiveness based on the information.

In some examples, IMD 14 does not necessarily sense a physiological parameter of patient 12. Instead, IMD 14 may control stimulation delivery based on parameters other than sensed physiological parameters. For example, IMD 14 may deliver stimulation during specific portions of the day, e.g., according to a schedule, or in response to patient activation, e.g., received via programmer 18.

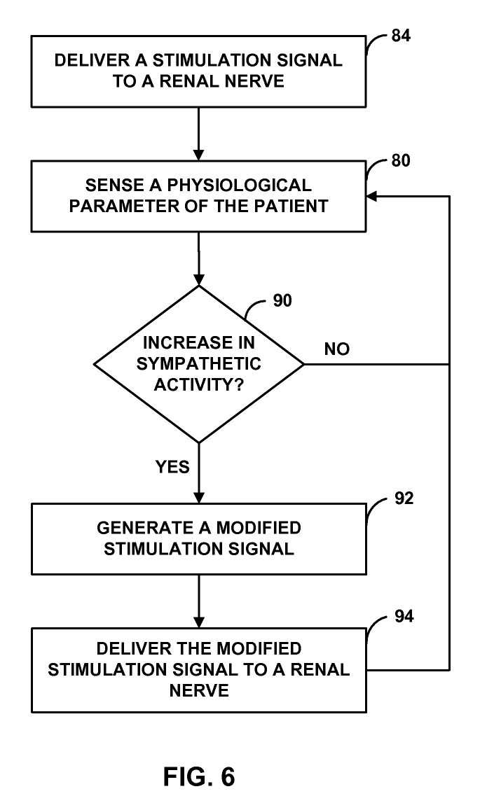

FIG. 6 is a flow diagram illustrating an example technique for modifying stimulation delivery based on a sensed physiological parameter. As described with respect to FIG. 5, IMD 14 may deliver a stimulation signal to one or more of renal nerves 28 (84), and sensing module 56 of IMD 14 may sense a physiological parameter of patient 12 (80). If IMD 14 identifies an increase in sympathetic activity within patient 12 based on the sensed physiological parameter (90), IMD 14 may generate a modified stimulation signal (92). For example, IMD 14 may modify one or more stimulation parameters, e.g., electrode configuration, amplitude, pulse width, and/or pulse rate, to increase the intensity of the stimulation signal in response to the detected increase in sympathetic activity. IMD 14 may deliver the modified stimulation signal to one or more of renal nerves 28 in response to the increase in sympathetic activity (94).

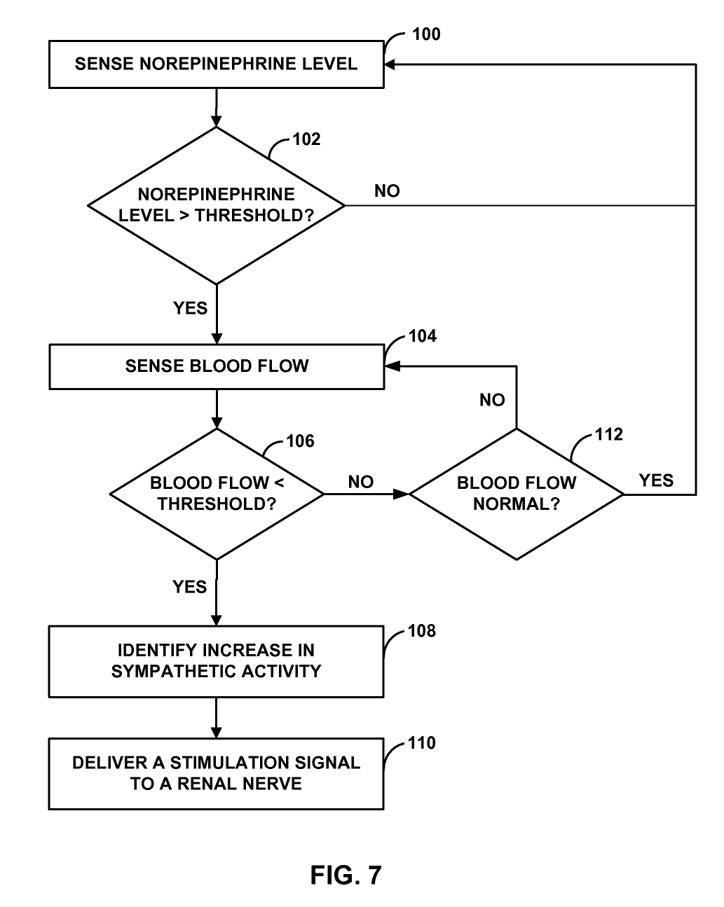

FIG. 7 is a flow diagram illustrating an example technique for sensing physiological parameters. IMD 14 may monitor a norepinephrine level with the patient's blood, e.g., within a renal vessel (100). Elevated norepinephrine levels may indicate elevated sympathetic activity. If the norepinephrine level rises above a threshold (102), IMD 14 may monitor renal blood flow, e.g., within renal arteries 32 (104). Since sympathetic efferent activation causes renal vasoconstriction and a reduction in renal blood flow, blood flow in a renal vessel may indicate the level of renal sympathetic activity. If blood flow to kidneys 20 is decreased below a threshold (106), IMD 14 may identify an increase in sympathetic activity within patient 12 (108). The sensed blood flow may confirm an increase in sympathetic activity detected by the norepinephrine level. For example, IMD 14 may only identify an increase in sympathetic activity when both norepinephrine and blood flow indicate increased sympathetic activity.

IMD 14 may deliver a stimulation signal to renal nerves 28 in response to the increase in sympathetic activity (110). For example, if IMD 14 was not previously delivering stimulation, IMD 14 may initiate stimulation delivery. If IMD 14 was already delivering stimulation therapy, IMD 14 may modify the stimulation signal, as described with respect to FIG. 6.

If blood flow to kidneys 20 is not decreased below a threshold (106), IMD 14 may determine if blood flow to kidneys 20 is normal, e.g., within a specified range (112). If blood flow is outside of the acceptable range, e.g., below the acceptable range but not below the threshold, IMD 14 may continue to monitor blood flow (104). Once the sensed blood flow returns to normal, e.g., is within a specified range, IMD 14 may switch back to monitoring norepinephrine levels (100).

The technique illustrated in FIG. 7 is merely an example. In general, IMD 14 may identify changes in the sympathetic activity level of patient 12 based on one or more sensed physiological parameters and control stimulation delivery to renal nerves 28 in response to the identified changes. In some examples, the sensed physiological parameters indicate renal sympathetic activity, and IMD 14 identifies changes in renal sympathetic activity. In some examples, IMD 14 may maintain sympathetic activity below a threshold level by adjusting stimulation delivery based on the sensed sympathetic physiological parameters. IMD 14 may use the sensed physiological parameters to determine when patient 12 requires stimulation and the minimum level of stimulation required to maintain renal sympathetic activity below a desired level. IMD 14 may sense physiological parameters on the right and left sides of patient 12, e.g., proximate to kidneys 20A and 20B, and/or control stimulation delivery to the right and left sides of patient 12, e.g., renal nerves 28A and 28B, individually.

FIG. 8 is a block diagram illustrating a system 120 that includes an external device 122, such as a server, and one or more computing devices 124A-124N that are coupled to the IMD 14 and programmer 18 via a network 126, according to one example. In this example, IMD 14 uses telemetry module 58 (FIG. 3) to communicate with programmer 18 via a first wireless connection, and to communicate with an access point 128 via a second wireless connection. In the example of FIG. 8, access point 128, programmer 18, external device 122, and computing devices 124A-124N are interconnected, and able to communicate with each other, through network 126.

In some cases, one or more of access point 128, programmer 18, external device 122, and computing devices 124A-124N may be coupled to network 126 through one or more wireless connections. IMD 14, programmer 18, external device 122, and computing devices 124A-124N may each comprise one or more processors, such as one or more microprocessors, DSPs, ASICs, FPGAs, programmable logic circuitry, or the like, that may perform various functions and operations, such as those described herein.

Access point 128 may comprise a device that connects to network 126 via any of a variety of connections, such as telephone dial-up, digital subscriber line (DSL), or cable modem connections. In other examples, access point 128 may be coupled to network 126 through different forms of connections, including wired or wireless connections. In some examples, access point 128 may communicate with programmer 18 and/or IMD 14. Access point 128 may be co-located with patient 12 (e.g., within the same room or within the same site as patient 12) or may be remotely located from patient 12. For example, access point 128 may be a home monitor that is located in the patient's home or is portable for carrying with patient 12.

During operation, IMD 14 may collect, measure, and store various forms of diagnostic data. For example, as described previously, IMD 14 may collect information regarding physiological parameters sensed via electrode 38, 40 and/or sensors 39. In certain cases, IMD 14 may directly analyze collected diagnostic data and generate any corresponding reports or alerts. In some cases, however, IMD 14 may send diagnostic data to programmer 18, access point 128, and/or external device 122, either wirelessly or via access point 128 and network 126, for remote processing and analysis.

For example, IMD 14 may send programmer 18 collected physiological parameter values indicative of sympathetic activity, which is then analyzed by programmer 18. Programmer 18 may generate reports or alerts after analyzing physiological parameter values and determine whether the values indicate that patient 12 requires medical attention, e.g., based on the physiological parameter values exceeding a threshold value. In some cases, IMD 14 and/or programmer 18 may combine all of the diagnostic data into a single displayable sympathetic activity report, which may be displayed on programmer 18. The sympathetic activity report may contain information concerning the physiological parameter measurements, the time of day at which the measurements were taken, and identify any patterns in the physiological parameter measurements. A clinician or other trained professional may review and/or annotate the sympathetic activity report, and possibly identify any patient conditions (e.g., heart disease).

In another example, IMD 14 may provide external device 122 with collected physiological parameter data via access point 128 and network 126. External device 122 includes one or more processors 130. In some cases, external device 122 may request collected physiological parameter data, and in some cases, IMD 14 may automatically or periodically provide such data to external device 122. Upon receipt of the physiological parameter data via input/output device 132, external device 122 is capable of analyzing the data and generating reports or alerts upon determination that the physiological parameter data indicates a patient condition may exist.

In one example, external device 122 may combine the diagnostic data into a physiological parameter report. One or more of computing devices 124A-124N may access the report through network 126 and display the report to users of computing devices 124A-124N. In some cases, external device 122 may automatically send the report via input/output device 132 to one or more of computing devices 124A-124N as an alert, such as an audio or visual alert. In some cases, external device 122 may send the report to another device, such as programmer 18, either automatically or upon request. In some cases, external device 122 may display the report to a user via input/output device 132.

In one example, external device 122 may comprise a secure storage site for diagnostic information that has been collected from IMD 14 and/or programmer 18. In this example, network 126 may comprise an Internet network, and trained professionals, such as clinicians, may use computing devices 124A-124N to securely access stored diagnostic data on external device 122. For example, the trained professionals may need to enter usernames and passwords to access the stored information on external device 122. In one example, external device 122 may be a CareLink server provided by Medtronic, Inc., of Minneapolis, Minn.

The techniques described in this disclosure, including those attributed to IMD 14, programmer 18, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various examples have been described. Although described primarily in the context of bilateral leads and stimulation, some examples include a single lead that provides unilateral stimulation of renal nerves proximate to one of the kidneys. These and other examples are within the scope of the following claims.

1. A method comprising:
sensing a physiological parameter of a patient;
generating a stimulation signal with an implantable electrical stimulator based on the physiological parameter;
delivering the stimulation signal from the implantable electrical stimulator to a renal nerve of the patient;
transmitting information regarding the physiological parameter to an external device outside of the patient; and
presenting the information to a user.

2. The method of claim 1, wherein delivering the stimulation signal to the renal nerve of the patient comprises delivering the stimulation signal to a left renal nerve on a left side of the patient and a right renal nerve on a right side of the patient.

3. The method of claim 2, wherein the stimulation signal comprises a first stimulation signal for delivery to the left renal nerve and a second stimulation signal for delivery to the right renal nerve, wherein the second stimulation signal differs from the first stimulation signal.

4. The method of claim 1, wherein presenting the information to the user comprises presenting information regarding at least one of blood pressure, heart failure status, or renal function to the user.

5. The method of claim 1, wherein sensing the physiological parameter comprises sensing a physiological parameter indicative of sympathetic activity within the patient, the method further comprising identifying an increase in sympathetic activity based on the physiological parameter, and wherein generating the stimulation signal comprises generating the stimulation signal in response to the increase in sympathetic activity.

6. The method of claim 1, wherein generating the stimulation signal comprises generating a biphasic stimulation signal with a frequency of approximately 100 hertz to approximately 20 kilohertz.

7. A system comprising:
an implantable sensor that senses a physiological parameter of a patient;
an implantable electrical stimulator that communicates with the implantable sensor, wherein the implantable electrical stimulator comprises a stimulation generator that generates a stimulation signal based on the physiological parameter and delivers the stimulation signal to a renal nerve of the patient; and
an external device that receives information regarding the physiological parameter and presents the information to a user.

8. The system of claim 7, further comprising a lead coupled to the implantable electrical stimulator, wherein the lead carries the implantable sensor and, wherein the implantable electrical stimulator delivers the stimulation signal to the renal nerve via one or more electrodes carried by the lead.

9. The system of claim 7, wherein the information comprises information regarding at least one of blood pressure, heart failure status, and renal function.

10. The system of claim 7, wherein the physiological parameter is indicative of sympathetic activity within the patient, the system further comprising a processor that identifies an increase in sympathetic activity based on the physiological parameter, and controls the implantable electrical stimulator to generate the stimulation signal in response to the increase in sympathetic activity.

11. The system of claim 7, wherein the stimulation signal comprises a biphasic stimulation signal with a frequency of approximately 100 hertz to approximately 20 kilohertz.

12. A method comprising:
sensing a physiological parameter indicative of sympathetic activity within a patient;
identifying an increase in sympathetic activity based on the physiological parameter; and
delivering a stimulation signal to a renal nerve of the patient in response to the increase in sympathetic activity.

13. The method of claim 12, wherein the physiological parameter is indicative of renal activity.

14. The method of claim 12, wherein sensing the physiological parameter comprises sensing the physiological parameter via a sensor positioned proximate to a renal nerve.

15. The method of claim 12, wherein the physiological parameter comprises at least one of blood pressure, blood flow, vascular tone, plasma renin level, or norepinephrine level.

16. The method of claim 12, wherein sensing the physiological parameter comprises sensing a first physiological parameter, the method further comprising sensing a second physiological parameter when the first physiological parameter indicates increased sympathetic activity, wherein identifying the increase in sympathetic activity comprises identifying the increase in sympathetic activity based on the first and second physiological parameters.

17. The method of claim 12, wherein the stimulation signal comprises a second stimulation signal, the method further comprising delivering a first stimulation signal to the renal nerve of the patient, wherein delivering the stimulation signal to the renal nerve of the patient in response to the increase in sympathetic activity comprises modifying the first stimulation signal in response to the increase in sympathetic activity to generate the second stimulation signal and delivering the second stimulation signal to the renal nerve of the patient.

18. The method of claim 12, further comprising:
transmitting information regarding the physiological parameter to an external device outside of the patient; and
presenting the information to a user.

19. The method of claim 12, wherein delivering the stimulation signal comprises delivering a biphasic stimulation signal with a frequency of approximately 100 hertz to approximately 20 kilohertz.

20. A system comprising:
a sensor that senses a physiological parameter indicative of sympathetic activity within a patient;
a processor that identifies an increase in sympathetic activity based on the physiological parameter; and
an electrical stimulator that delivers a stimulation signal to a renal nerve of the patient in response to the increase in sympathetic activity.

21. The system of claim 20, wherein the processor comprises a processor of the electrical stimulator.

22. The system of claim 20, further comprising a lead, wherein the electrical stimulator delivers the stimulation signal to the renal nerve via one or more electrodes carried by the lead.

23. The system of claim 22, wherein the lead carries the sensor.

24. The system of claim 20, wherein the electrical stimulator comprises an implantable electrical stimulator.

25. The system of claim 20, further comprising:
a telemetry module that transmits information regarding the physiological parameter to an external device outside of the patient; and
the external device that presents the information to a user.

26. The system of claim 20, wherein the stimulation signal comprises a biphasic stimulation signal with a frequency of approximately 100 hertz to approximately 20 kilohertz.

27. A method inhibiting renal autonomic activity comprising:
generating a biphasic stimulation signal with a frequency of approximately 100 hertz to approximately 20 kilohertz; and
delivering the stimulation signal to a renal nerve of the patient.

28. The method of claim 27, wherein generating the biphasic stimulation signal comprises generating the biphasic stimulation signal with a frequency of approximately 100 hertz to approximately 10 kilohertz.

29. The method of claim 27, wherein generating the biphasic stimulation signal comprises generating the biphasic stimulation signal with a frequency greater than approximately 2 kilohertz.

30. The method of claim 27, wherein generating the stimulation signal comprises generating the stimulation signal with an amplitude of approximately 0.5 volts to approximately 10 volts.

31. The method of claim 27, further comprising sensing a physiological parameter of the patient, wherein generating the stimulation signal comprises generating the stimulation signal based on the physiological parameter.

32. The method of claim 31, further comprising:
transmitting information regarding the physiological parameter to an external device outside of the patient; and
presenting the information to a user.

33. The method of claim 31, wherein sensing the physiological parameter comprises sensing a physiological parameter indicative of sympathetic activity within the patient, the method further comprising identifying an increase in sympathetic activity based on the physiological parameter, and wherein generating the stimulation signal comprises generating the stimulation signal in response to the increase in sympathetic activity.

34. The method of claim 27, wherein delivering the stimulation signal comprises delivering the stimulation signal according to a schedule.

35. A system comprising:
means for generating a biphasic stimulation signal with a frequency of approximately 100 hertz to approximately 20 kilohertz; and
means for delivering the stimulation signal to a renal nerve of the patient.

36. A system comprising:
a signal generator that generates a biphasic stimulation signal with a frequency of approximately 100 hertz to approximately 20 kilohertz; and
an electrode configured to be positioned proximate to a renal nerve of the patient, wherein the signal generator delivers the stimulation signal to the renal nerve via the electrode.

37. The system of claim 36, wherein the biphasic stimulation signal comprises a frequency of approximately 100 hertz to approximately 10 kilohertz.

38. The system of claim 36, wherein the biphasic stimulation signal comprises a frequency greater than approximately 2 kilohertz.

39. The system of claim 36, further comprising a sensor that senses a physiological parameter of the patient, wherein the signal generator generates the stimulation signal based on the physiological parameter.

40. The system of claim 39, further comprising:
a telemetry module that transmits information regarding the physiological parameter to an external device outside of the patient; and
the external device that presents the information to a user.

41. The system of claim 39, wherein the physiological parameter is indicative of sympathetic activity within the patient, the system further comprising a processor that identifies an increase in sympathetic activity based on the physiological parameter, and wherein the signal generator generates the stimulation signal in response to the increase in sympathetic activity.

42. The system of claim 36, further comprising a lead coupled to the signal generator, wherein the lead carries the electrode.

43. The system of claim 42, wherein the lead is implanted within a renal vessel of the patient.

44. The system of claim 36, wherein the signal generator delivers the stimulation signal according to a schedule.
